(19) United States
(12) Patent Application Publication
(10) Pub. No.: US 2005/0171169 A1
(43) Pub. Date: Aug. 4, 2005

(54) Combination chemotherapy for helminth infections

(76) Inventor: Jean Francois Rossignol, Clearwater, FL (US)

Correspondence Address: PENDORF & CUTLIFF, 5111 Memorial Highway, Tampa, FL, 33634-7356, US

(21) Appl. No.: 10/770,274

(22) Filed: Feb. 2, 2004

(51) Int. Cl. A61K 31/426; A61K 31/4184

(52) U.S. Cl. 514/370; 514/394

(57) ABSTRACT

Methods for the treatment of a helminthic infection, by the administration, in either single or multiple dosages, of a synergistic combination comprising albendazole according to formula (I): 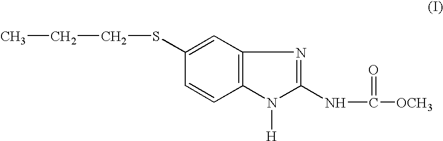
and nitazoxanide according to formula (II): 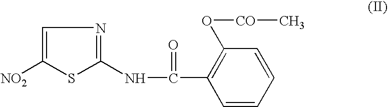 in which the efficacy and range of parasites treated is enhanced by the instant combination compared to the sum of the effects of the subject drugs administered separately.

### FIELD OF THE INVENTION

The invention relates to compositions and methods for the treatment and prevention of larval and adult stage helminth infection, in which the helminth infection is a nematode, cestode, or trematode infection. More specifically, the invention relates to administration of a synergistic combination of albendazole (ABZ) and nitazoxanide (NTZ), which is effective for single dose treatment of a broad spectrum of intestinal helminth infections, and is also effective for repeated dose treatment of the larval stage of helminths such as Echinococcus multilocularis, Echinococcus granulosis, and Cysticercus cellulosae.

### BACKGROUND OF THE INVENTION

Helminth infections are a significant public health problem worldwide. Intestinal helminthes, for example, chronically affect about one-third of the world's population, with an estimated prevalence of one billion cases of roundworm, 900 million cases of trichuriasis, and 500 million cases of hookworm. Parasitic infections disproportionately affect school-age children and are often transmitted where sanitation is poor. Further, even when successfully treated, re-infection is common. Compounding this problem, humans and animals may act as asymptomatic carriers.

Mass treatment programs for intestinal helminthic diseases have been proposed and implemented in developing countries to attempt to break the cycle of infection, reduce the number of helminthes in the population, and thereby prevent infection and re-infection. Entire communities that are at risk have been treated with anti-helminthic drugs such as albendazole or praziquantel. However, a drawback related to use of these drugs for mass treatment is that they are effective against a relatively small number of the intestinal helminthes that infect humans or animals.

An ideal anti-helminthic drug, or combination of drugs, that is suitable for mass treatment of intestinal helminth infections, would be safe and effective against a broad spectrum of helminthes. In addition, a preferred drug or combination of drugs would be effective when given as a single dose, to avoid difficulties associated with drug supply and compliance. Combinations of drugs may beneficially minimize the emerging problem of drug-resistance. A preferred combination of drugs should also exhibit synergy between the drugs such that the combination is more effective than the effects of the drugs when given separately. Unfortunately, within the prior art, no such ideal formulation currently exists.

While combinations of drugs, such as praziquantel and albendazole, have been tested, they lack one or more of these ideal properties. For example, clinical trials of a combination of albendazole and praziquantel against Trichuris trichuria infection showed no synergistic interaction between the two drugs (Sirivichayakul, C. et al., Southeast Asian J. Trop. Med. Public Health. 2001 32: 297-301). Synergism was also not observed in the treatment of geohelminths and schistosomiasis in schoolchildren by combined albendazole and praziquantel treatment (Olds, G. R. et al. 1999, J. Infect. Diseases 179: 996-1003) in vivo treatment of E. multilocularis in rats (Taylor, D. H., Morris, D. L., Richards, K. S. and Reffin, D. 1988. Echinococcus Multilocularis: in vivo results of therapy with albendazole and praziquantel, Trans. R. Soc. Trop. Med. Hyg. 82: 611-5).

In addition to mass treatment of intestinal helminthic infections, there is also a need for drugs or combinations of drugs that are safe and effective in the treatment of certain larval stage helminthic diseases that have proved difficult to treat by other means, such as the cestode diseases caused by Echinococcus multilocularis, Echinococcus granulosis, and Cysticercus cellulosae.

Cysticercus cellulosae is the intermediate stage of the tapeworm Taenia solium, which occurs in the small intestine of humans. Cysticerci are found in the brain, liver, heart and skeletal muscles, and cause an inflammatory response in the muscle and central nervous system. Humans infection results from ingestion of cysts of T. solium eggs or from auto-infection via an adult worm in the intestine. Human subjects may develop seizures, headache, nausea, vomiting, inability to walk, poor vision due to enlargement of the brain, and increased pressure in the brain. Other neurological problems may develop depending on the location of the cysts. An estimated 50-100 million people are infected worldwide, primarily in Latin America, India, China, Southeast Asia, and sub-Saharan Africa. The drugs praziquantel and albendazole are currently used to treat cysticercosis, but these drugs are not effective in all patients, such as those with calcified cysts or brain enlargement, and improved drugs or drug combinations are urgently required.

Hydatid disease is caused by cyst-like larvae of Echinococcus granulosis and is endemic in many parts of the world. The liver and lung are the most frequently affected organs. Large lesions (1-15 cm) form in the liver or lung, causing pain and occasionally rupturing. No fully effective chemotherapeutic agent is presently available for the medical management of hydatid disease, and improved drugs or combinations of drugs are required. At present, treatment typically comprises administration of praziquantel, albendazole, or more recently in animal studies, oxfendazole (Ronald E. Blanton et al. “Oxfendazole Treatment for Cystic Hydatid Disease in Naturally Infected Animals” Antimicrob. Agents Chemother. (1998) 42 (3): 601-605). Clinical trials of the effectiveness of mebendazole, flubendazole and albendazole administered separately showed limited success, and it was concluded that new drugs, formulations and/or forms of application were needed (Davis, A., Pawlowski, Z. S. and Dixon, H. “Multicentre clinical trials of benzimidazolecarbamates in human echinococcosis” Bull. W. H. O., 64: 383-388, 1986).

Improved drugs or drug combinations are also needed that are effective against Alveolar echinococcosis (AE), which is caused by the metacestode (larval) stage of Echinococcus multilocularis. AE is a life-threatening disease confined to the northern hemisphere (Eckert, J., and P. Deplazes. 1999. Alveolar echinococcosis in humans: the current situation in central Europe and the need for countermeasures. Parasitol. Today 15: 315-319; Eckert, J., F. J. Conraths, and K. Tackmann. 2000. Echinococcosis—an emerging or re-emerging zoonosis. Int. J. Parasitol. 30: 1283-1294). The adult tapeworm exists as an enteric parasite in certain carnivores, and parasite eggs are shed in the feces. Each egg comprises an oncosphere, which upon ingestion by a suitable intermediate host and subsequent passage through stomach and intestine becomes activated, penetrates the mucosa, enters blood and lymphatic vessels, and enters the liver. In the liver parenchyma, oncospheres develop over time to form mature metacestodes, which are characterized by tumor-like proliferation. Metastases formation in other organs has also been reported (Mehlhorn, H., J. Eckert, and R. C. A. Thompson. 1983. Proliferation and metastases formation of larval Echinococcus multilocularis. It. Ultrastructural investigations. Z. Parasitenkd. 69: 749-763).

The anti-helminthic drugs presently used for treatment of AE are the benzimidazole-derivatives mebendazole (MBZ) and albendazole (ABZ) (Reuter, S., B. Jensen, K. Buttenschoen, W. Kratzer, and P. Kern. 2000. Benzimidazoles in the treatment of alveolar echinococcosis: a comparative study and review of the literature. J. Antimicrob. Chemother. 46: 451-456; Schantz, P. M., H. van den Bossche, and J. Eckert 1982. Chemotherapy of larval echinococcosis in animals and humans. Report of a workshop. Z Parasitenkunde 67: 5-26; Wilson, J. F., and R. L. Rausch. 1982. Mebendazole and alveolar hydatid disease. Annals of Tropical Medicine and Parasitology 76: 165-173; and Wilson, J. F., M. Davidson, and R. L. Rausch. 1978. A clinical trial of mebendazole in the treatment of alveolar hydatid disease. American Review of Respiratory Disease 118: 747-757). Chemotherapy employing these drugs has been successful in many cases in effectively stopping the growth of the parasite.

However, failures in benzimidazole treatments have been reported, either related to severe side effects such as liver toxicity, which typically leads to discontinuation of treatment (Davis, A., Z. S. Pawloski, and H. Dixon. 1986. Multicentre clinical trials of benzimidazolecarbamates in human echinococcosis. Bull. W. H. O. 64: 383-388), or to progressive disease despite benzimidazole treatment, observed in approximately 16% of all cases. In humans, numerous side effects of benzimidazoles have been described, including gastrointestinal disturbances, reversible alopecia, elevation of serum transaminases, proteinuria, neurological symptoms, and neutropenia (Reuter, S., B. Jensen, K. Buttenschoen, W. Kratzer, and P. Kern. 2000. Benzimidazoles in the treatment of alveolar echinococcosis: a comparative study and review of the literature. J. Antimicrob. Chemother. 46: 451-456).

Efficacy and dosage of benzimidazole treatment is further limited because, beyond a certain dosage, outcome is not significantly improved (Ramalingam, S., Sinniah, B. & Krishnan, U., Am J. Trop. Med. Hyg. (1983) 32: 984-9). Also, benzimidazoles do not appear to be helminthicidal in vivo, and the helminth may resume growth after discontinuation of treatment (Vuitton D. A. 2003. The ambiguous role of immunity in echinococcosis: protection of the host or of the parasite? Acta Trop. 85: 119-132). Due to these constraints, many patients have to take the drugs on a lifelong basis to prevent recurrence of AE.

Therefore, novel treatment options for AE, and other cestode diseases, are urgently needed. At present, there are no reliable chemotherapeutic alternatives to benzimidazoles for the treatment of AE. Other drugs have been tested, such as praziquantel and alpha-difluoromethylornithine in the animal model, but with limited success (Miyaji, S., K. Katakura, S. Matsufuji, Y. Murakami, S. Hayashi, Y. Oku, M. Okamato, and M. Kamiya. 1993. Failure of treatment with alpha-difluoro-methylornithine against secondary multilocular echinococcosis in mice. Parasitol. Res. 79: 75-76; Marchiondo, A. A., R. Ming, F. L. Andersen, J. H. Slusser, and G. A. Conder. 1994. Enhanced larval cyst growth of Echinococcus multilocularis in praziquantel-treated jirds (Meriones unguiculatus). Am. J. Trop. Med. Hyg. 50: 120-127).

We have recently reported on the in vitro helminthicidal activity of nitazoxanide (2-acetolyloxy-N-(5-nitro 2-thiazolyl)benzamide; NTZ) against E. multilocularis metacestodes (Stettler, M., R. Fink, M. Walker, B. Gottstein, T. Geary, J. F. Rossignol, and A. Hemphill. 2003. In vitro parasiticidal effect of nitazoxanide against Echinococcus multilocularis metacestodes. Antimicrob Agents Chemother. 47: 467-74). NTZ was originally developed as a veterinary antihelminthic, and was first described in 1984 as a human cestocidal drug (Rossignol, J. F., and H. Maisonneuve. 1984. Nitazoxanide in the treatment of Taenia saginata and Hymenolepis nana. Am. J. Trop. Med. Hyg. 33: 511-512). No serious adverse side effects in humans have been described to date.

NTZ is also known as a broad-spectrum drug against a wide variety of intestinal parasites and enteric bacteria infecting animals and humans (reviewed in: Gilles, H. M., and P. S. Hoffman P. S. 2002. Treatment of intestinal parasitic infections: a review of nitazoxanide. Trends Parasitol. 18: 95-97). NTZ exhibits structural similarities to ABZ and its metabolic derivatives albendazole sulfoxide (ABZSO) and albendazole sulfone (ABZSN), with a 5-nitrothiazole ring substituting the benzimidazole ring. In anaerobic bacteria and protozoa, NTZ interferes with PFOR-dependent electron transfer reactions that are essential for anaerobic energy metabolism. The metabolic products are tizoxanide (TIZ) and tizoxanide-glucuronide (TIZ-glue). Nothing is currently known regarding the possible mode of action of NTZ for helminthes, however, the enzymes of anaerobic electron transport must be considered as potential targets.

In view of the deficiencies noted above in the prior art, there is a need for safe drugs or combinations of drugs that exhibit broad spectrum intestinal anti-helminthic activity in humans and animals, in which the activities of the drugs at least support each other and are preferably synergistic, and that are effective both for single dose treatment of a broad spectrum of intestinal helminth infections, and also are effective for repeated dose treatment of the larval stage of helminths such as Echinococcus multilocularis, Echinococcus granulosis, and Cysticercus cellulosae. These benefits and more are set forth in the following description.

### SUMMARY OF THE INVENTION

The present invention is based on the surprising discovery of a synergistic interaction between albendazole [5-(propylthio)-1H-benzimidazol-2-yl)]carbamic acid methyl ester, and nitazoxanide (2-(acetolyloxy)-N-(5-nitro-2-thiazolyl)benzamide), whereby the anti-helminthic activity of the combination is greater than the activities of the compounds when administered separately, and the combination is safe for administration to mammals. The synergism between albendazole and nitazoxanide exhibits as enhanced activity against helminthic infections, such as nematodes, cestodes and trematodes, and also against specific parasites, such as Echinococcus granulosis or Echinococcus multilocularis, for which existing drugs are either poorly effective or not effective at all.

Not to be limited by theory, the basis of the synergy between albendazole and nitazoxanide may result from the increase in serum levels of their corresponding bio-active metabolites when the two drugs are administered in combination or at similar times.

Thus, in a first aspect, the present invention provides a combination chemotherapy for helminthic diseases including nematode, cestode, and trematode infections, and which has an improved spectrum of anti-helminthic activities that is for the first time suitable for single dose mass chemotherapy of intestinal helminthes, and for multiple dose treatment of larval stages of helminthes such as Echinococcus multilocularis, Echinococcus granulosis, and Cysticercus cellulosae.

In this first aspect, the method for the treatment of one or more helminthic infections in a human or in an animal subject includes administering to the subject an efficacious amount of a composition comprising albendazole ([5-(propylthio)-1H-benzimidazol-2-yl)]carbamic acid methyl ester, formula (I)): 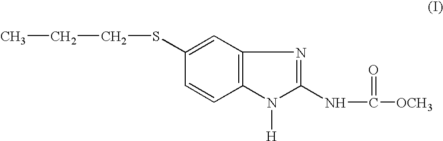
and nitazoxanide (2-(acetolyloxy)-N-(5-nitro-2-thiazolyl)benzamide, formula (II)): 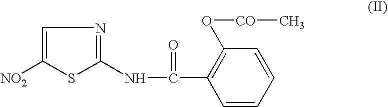
   wherein these formulae are to be understood to include all of their pharmaceutically acceptable salts, and the composition may include one or more pharmaceutically acceptable carriers.

The pharmaceutical composition can be in a form suitable for oral administration, as a solid dosage form, a liquid suspension, or a paste.

In a second aspect, the present invention provides a method for improving the efficacy of albendazole in a subject administered albendazole through increasing the serum albendazole sulfoxide levels in the subject by administering an effective amount of a composition comprising nitazoxanide, or a pharmaceutically acceptable salt of nitazoxanide, in a pharmaceutically acceptable carrier.

In a third aspect, the present invention provides a method for reducing metacestode formation in a subject by administering to the subject an effective amount of albendazole [5-(propylthio)-1H-benzimidazol-2-yl)]carbamic acid methyl ester and nitazoxanide (2-(acetolyloxy)-N-(5-nitro-2-thiazolyl)benzamide), or any pharmaceutically acceptable salts thereof, in a pharmaceutically acceptable carrier, either separately or in combination.

The foregoing has outlined rather broadly the more important features of the present invention in order that the detailed description of the invention that follows may be better understood and so that the present contribution to the art can be more fully appreciated. Additional features of the invention will be described hereinafter, which form the claims of the invention. It should be appreciated by those skilled in the art that the conception and the specific embodiments disclosed may be readily utilized as a basis for modifying or designing other pharmaceutical compositions and methods for treatment for carrying out the same purposes of the present invention. It should also be realized by those skilled in the art that such equivalent formulations and methods do not depart from the spirit and scope of the invention as set forth in the appended claims.

### BRIEF DESCRIPTION OF THE FIGURES

FIG. 1 Shows the effects of albendazole and nitazoxanide treatments, separately and in the combination of the present invention, on secondary E. multiliocularis infection, with drug treatment starting at 2 months post infection The experiment was carried out with 10 animals in each experimental group, and results are presented as means +/−standard deviation.

FIG. 2 Shows histological sections of metacestode tissue of Example 2 stained with methylene blue-azurII/basic fuchsin. (A) untreated tissue, (B) NTZ/ABZ combination treatment. Large arrows in (B) indicates densely encapsulated laminated layer. GL=germinal layer; LL=laminated layer; host=host connective tissue.

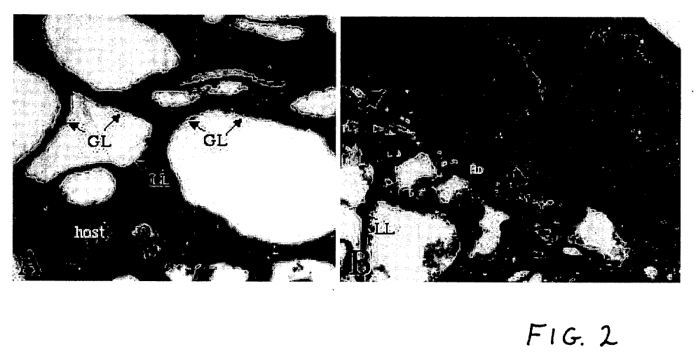

FIG. 3 Shows transmission electron microscopic analysis of drug efficacy. (A) CMC-treated control, showing a large number of long microtriches protruding substantially into the laminated layer, as indicated by arrows. (B) and (C) show alterations induced by ABZ treatment, and (D) and (E) show parasite tissue obtained from NTZ-treated mice. GL=germinal layer, LL=laminated layer, gc=glycogen storage cell, uc=undifferentiated cell. (A) bar=2.2 μm, (B) bar=2.1 μm, (C) bar=2.2 μm, (D) bar=3.1 μm, (E) bar=0.7 μm.

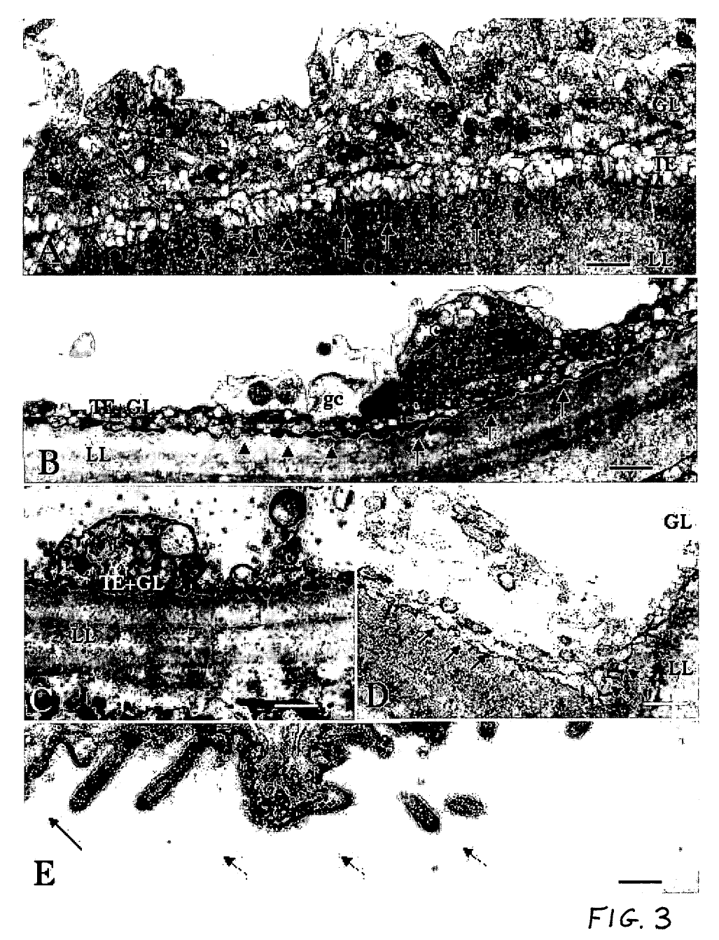

FIG. 4 Shows Transmission Electron Microscopy (TEM) of metacestodes undergoing combined NTZ/ABZ treatment. (A) showing complete regression of metacestode germinal layer, with only the tegument (TE) constituting the viable parasite tissue, and complete absence of microtriches (arrows), (B) largely damaged undifferentiated cell (uc) and residues of glycogen storage cell (gc), (C) laminated layer (LL) with microtrichal residues (mt) and accumulation of small vesiculate structures (arrows) embedded into the matrix of the laminated layer, (D) sheet of laminated layer lacking any parasite tissue, completely encapsulated in host tissue. host=host connective tissue. (A) bar=4.1 μm, (B) bar=2.1 μm,(D) bar=0.5 μm, (E) bar=2.6 μm.

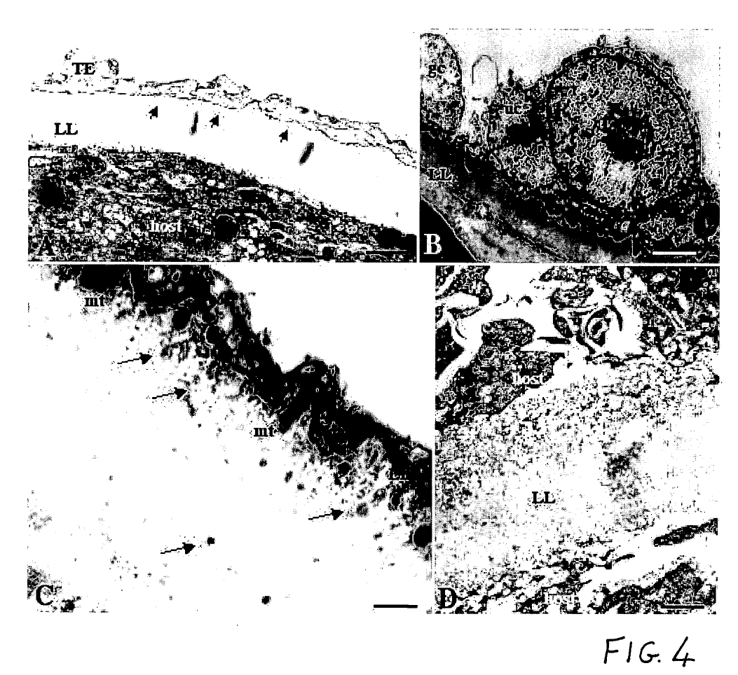

FIG. 5. Shows the pharmacokinetics following uptake of a single dose of albendazole, nitazoxanide, and the combination of albendazole and nitazoxanide of present invention. Serum levels of the nitazoxanide metabolites tizoxanide (TIZ) (A), tizoxanide-glucuronide (TIZ-gluc) (B), and albendazole sulfoxide (ABZSO) (C) are shown. Results are presented as the means of three mice+/−SD.

### DETAILED DESCRIPTION

The method for treatment of a broad range of helminthic infections in a human or an animal subject comprises administering an effective amount of a composition comprising albendazole ([5-(propylthio)-1H-benzimidazol-2-yl)]carbamic acid methyl ester, formula (I)): 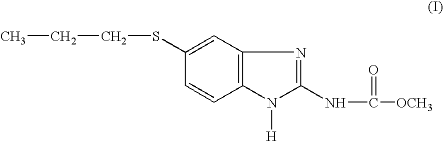
and nitazoxanide (2-(acetolyloxy)-N-(5-nitro-2-thiazolyl)benzamide, formula (II)): 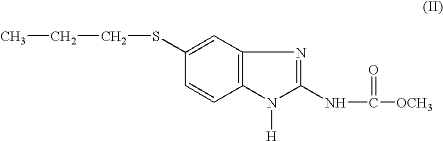
or pharmaceutically acceptable salts thereof, in a pharmaceutically acceptable carrier.

Albendazole, the compound of formula (I), sometimes referred to herein as ABZ, is the generic name for [5-(propylthio)-1H-benzimidazol-2-yl)]carbamic acid methyl ester. The preparation and certain uses of albendazole are disclosed in U.S. Pat. No. 3,915,986. Albendazole has a molecular weight of 265.34, and a melting point of 208-210° C.

Compositions comprising albendazole are preferably formulated for oral administration and may therefore take the form of a liquid, for example an emulsion or a solution or a suspension in water or oil such as arachis oil, or other liquid. Formulation of non-aqueous micellar solutions of albendazole may be prepared according to the method disclosed in U.S. Pat. No. 5,169,846.

Nitazoxanide, the compound of formula (II), sometimes referred to hereafter as NTZ or compound PH 5776, is the generic name for 2-acetolyloxy)-N-(5-nitro 2-thiazoly)benzamide, a compound first synthesized by Rossignol and Cavier in 1975 and subsequently shown to have activity against a number of protozoan and helminthic pathogens (reviewed in: Gilles, H. M., and P. S. Hoffman P. S. 2002. Treatment of intestinal parasitic infections: a review of nitazoxanide. Trends Parasitol. 18: 95-97). NTZ has a molecular weight of 307.2, presents as odorless yellow granules with a melting point of 202-204° C.; is very poorly soluble in water, ether and methyl benzene; is poorly soluble in ethanol, chloroform and acetic acid; is fairly soluble in dioxane and acetone, and is easily soluble in pyridine. Solubilization in DMSO is recommended: the solubility of NTZ in DMSO is at least 2 mg/mL, and NTZ is easily absorbed orally.

The preparation and certain uses of NTZ are disclosed in U.S. Pat. No. 3,950,351 and WO 95/28393.

A composition comprising NTZ may be administered in either a solid dosage form or an aqueous suspension, and it is preferred that the composition contain the effective dose of the active agent in the form of solid particles having a particle size of smaller than 200 μm. Preferably, the particle size is between 10 and 100 μm, and most preferably between 20 and 50 μm. The means for measuring particle size is disclosed in U.S. Pat. No. 5,856,348.

The present invention relates to pharmaceutical compositions comprising ABZ and NTZ, and may advantageously further comprise at least one pharmaceutically acceptable acid. Examples of such acids are citric acid, glutamic acid, succinic acid, ethanesulfonic acid, acetic acid, tartaric acid, ascorbic acid, methanesulfonic acid, fumaric acid, adipic acid, malic acid, and mixtures thereof. Citric acid is preferred. The presence of acid improves stability of the active ingredients.

The compositions of the present invention may be formulated as a solid or liquid dosage form, or as pastes or ointments, and may optionally contain further active ingredients such as additional anti-parasite compounds, or antibiotics, antiviral agents and the like.

The compositions of the present invention may comprise a pharmaceutically acceptable carrier, which is not particularly limited, and is known to include a wide range of carriers known to those of ordinary skill in the art, and may include wetting or dispersing agents (U.S. Pat. No. 5,578,621), starch derivatives (U.S. Pat. No. 5,578,621), excipients, and the like. Tablet embodiments may optionally comprise a coating of a substance that constitutes an enteric coating, i.e. a coating that substantially insoluble in gastric secretion but substantially soluble in intestinal fluids.

The compositions of the present invention may be administered as a single dose, or from 1 to 5 times daily, to an infected or susceptible subject for curative or preventative anti-helminth activity.

The preferred dosages of ABZ and NTZ in the compositions of the present invention may depend upon the weight of the subject, and may be those dosages that are well known in the art for the separate administration of ABZ or NTZ. Preferably, ABZ is administered in a range between about 200 and 800 mg, and for an adult human a dosage of 400 mg ALB is preferred. NTZ is administered in a range between about 100 and 3000 mg, and for an adult human a single dosage of about 2000 mg NTZ is most preferred.

The ratio of nitazoxanide:albendazole (w/w) is preferably from about 2:1 to about 8:1, and the ratio is most preferably 5:1.

It is important to note that it is not essential to the method of the present invention that NTZ and ABZ be administered in the same formulation. It is specifically contemplated that contemporaneous administration of separate formulations of NTZ and ABZ falls within the scope of the method of the present invention.

It is not required that NTZ and ABZ, if mixed, are administered as a single formulation. For example, a single dosage of 4 tablets, each comprising about 500 mg NTZ and about 100 mg ABZ is preferred. Such a single dosage is suitable for the treatment and prevention of intestinal helminth infections, but similar formulations may also be used for repeated dosage treatment of the larval stage of helminths such as Echinococcus multilocularis, Echinococcus granulosis, and Cysticercus cellulosae, as demonstrated by the continuing clinical studies directed to comparison of combined ABZ/NTZ treatment, versus separate ABZ or NTZ treatment, of helminthic diseases.

A combined ABZ/NTZ formulation suitable for use in the practice of the present invention comprises a core tablet and a coating as follows:

CORE TABLET:

| | | |
|---|---|---|
| NITAZOXANIDE | 500 | mg |
| ALBENDAZOL | 100 | mg |
| CORN STARCH | 60 | mg |
| CORN STARCH PREGELATINIZED | 85 | mg |
| HYDROXY-PROPYL-METHYL-CELLEULOSE | 16 | mg |
| GRANULATED SUGAR | 20 | mg |
| PRIMOGEL | 30 | mg |
| TALC | 8 | mg |
| MAGNESIUM STEARATE | 7 | mg |
| PURIFIED WATER (lost in manufacture) | 0.205 | ml |

COATING:

| | | |
|---|---|---|
| EUDRAGIT L 12.5 | 28.2 | mg |
| EUDRACOLOR YELLOW 085 | 84.6 | mg |
| ISOPROPYL ALCOHOL (lost in manufacture) | 0.055 | ml |
| KETONE (lost in manufacture) | 0.036 | ml |

The core tablet is manufactured, for example, by performing the following steps: wet granulation; drying; and compression. Film coating is performed with organic solvents.

In further detail, the core tablet is manufactured as follows:
1. The hydroxymethylcellulose is dissolved in purified water;
2. The nitazoxanide, albendazole, maize starch, pregelatinized starch and granulated sugar are mixed;
3. mixture 2 is wetted with solution 1, and purified water is added as required to obtain a complete wetting of the mixture 2;
4. granulate 3 is dried 60 degrees centigrade to 3.0 to 3.5% humidity;
5. granulate 4 is sieved through a mesh 12 sieve on an oscillating machine;
6. granulate 5 is mixed with primogel;
7. talc and magnesium stearate are mixed with granulate 6;
8. mixture 7 is compressed to the correct weight, friability and minimum hardness.

The coating is applied to the core tablet by preparing a coating suspension of Eudragit L 12.5 with isopropyl alcohol and acetone. Yellow Eudracolor 085 is added under stirring. During the coating process stirring is maintained. The cores are spray coated in a heated rotating coating drum and then dried.

The spectrum of parasitic diseases that may be treated or prevented by the method of the present invention includes those parasites known in the art to be separately treatable by ABZ or NTZ administration, including but not limited to, roundworm, hookworm, whipworm, and pinworm. Additionally, the method encompasses treatment of parasites that are more effectively treated by the combination of ABZ and NTZ, such as the larval stage of helminths such as Echinococcus multilocularis, Echinococcus granulosis, and Cysticercus cellulosae. Without being limited by theory, the basis for the synergistic activities of ABZ and NTZ may be the discovery that the combined application of NTZ and ABZ influences the serum levels of their corresponding bio-active metabolites. Thus, the synergism between NTZ and ABZ of the method of the present invention applies generally and is not restricted to the treatment only of specific parasites.

With respect to non-human animals, the present invention contemplates treatment of at least the following parasites: Haemonchus, Osteragia, Trichostrogylusi, Nematodirus, Cooperia, Bunostomum, Strongyloides, Trichuris, Oesophagostomum, Chabertia, Dicryocaulus, Moniezia and Fasciola in sheep. They are active against Haemonchus, Ostertagia, Trichostrongylus, Nematodirus, Cooperia, Bunostomum, Capillaria, Strongyloides, Trichuris, Oesophagostomum, Chabertia, Dictyocaulus, Moniezia and Fasciola in cattle.

### EXAMPLES

In the following five EXAMPLES, the surprising synergy of a combined NTZ/ABZ-treatment protocol for chemotherapy against alveolar echinococcosis (AE) is demonstrated.

In EXAMPLE 1, the relative ineffectiveness of albendazole or nitazoxanide chemotherapy against secondary AE, when administered separately, is shown.

In EXAMPLE 2, when treatment was initiated at 2 months p. i. and the parasite had sufficient time to establish itself within the host, the combined NTZ/ABZ-treatment protocol is shown to possess synergistic advantages compared to separate treatment with NTZ or ABZ.

In EXAMPLE 3, the efficacy of nitazoxanide chemotherapy was shown in the treatment of primary infection with E. multilocularis eggs. Thus, the combination ABZ/NTZ treatment includes the beneficial activity shown in EXAMPLE 3.

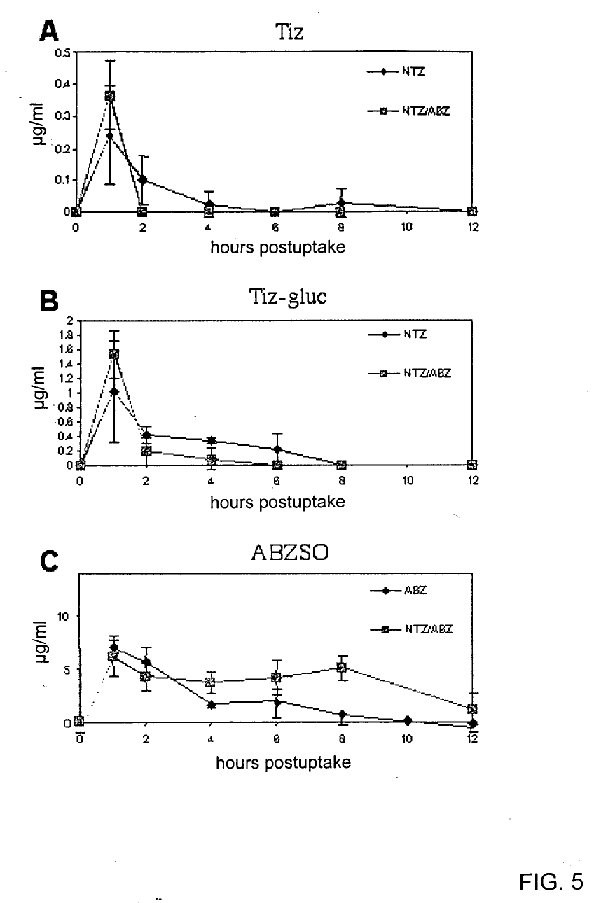

In EXAMPLE 4, a pharmacokinetic study shows that the combined application of NTZ and ABZ influences the serum levels of their corresponding bio-active metabolites, showing that the uptake of one drug influences the absorption of the other (see FIG. 5), resulting in surprising synergism between NTZ and ABZ. In particular, the combined chemotherapy enhanced ABZSO-serum levels (see FIG. 5C).

In EXAMPLE 5, the markedly increased efficacy of NTZ/ABZ combination chemotherapy is shown by histology and electron microscopy. Although NTZ and ABZ separately induced ultrastructural alterations in vivo, which had been previously reported upon in vitro treatment with ABZSO and NTZ, respectively (Ingold, K., P. Bigler, W. Thormann, T. Cavaliero, B. Gottstein, and A. Hemphill. 1999. Efficacies of albendazole sulfoxide and albendazole sulfone against in vitro cultivated Echinococcus multilocularis metacestodes. Antimicrob. Agents Chemother. 43: 1052-1061; and Stettler, M., R. Fink, M. Walker, B. Gottstein, T. Geary, J. F. Rossignol, and A. Hemphill. 2003. In vitro parasiticidal effect of nitazoxanide against Echinococcus multilocularis metacestodes. Antimicrob Agents Chemother. 47: 467-74).

However, a comparative investigation of these ultrastructural features shows a pronounced synergistic effect of combined ABZ/NTZ chemotherapy, as indicated by a high degree of structural damage. Most strikingly, the extensive degeneration of parasite tissue and the loss of viable germinal layer and progressive necrosis, often resulted in encapsulation of residual laminated layer by host connective tissue.

### Methods

E. multilocularis metacestodes (isolate IM280) were maintained by serial transplantation passages through intraperitoneal (i.p.) injection in gerbils (Meriones unguiculatus). Animals were sacrificed at four to ten weeks p.i, and the parasite material was removed from the peritoneal cavity under aseptic conditions, placed into Hanks balanced salt solution (HBSS), and washed several times. Vesicle suspensions were prepared as described in Hemphill, A., and B. Gottstein. 1995. Immunology and morphology studies on the proliferation of in vitro cultivated Echinococcus multilocularis metacestodes. Parasitol. Res. 81: 605-614. Vesicles were washed once in HBSS, and the number of intact metacestodes in 50 ml HBSS was determined microscopically. The metacestode suspension was adjusted to 20 intact vesicles/100 ml HBSS, and was used for infection experiments immediately.

Infective E. multilocularis eggs were isolated from fox intestinal tissue as described (Pater, C., V. Muller, S. Harraga, M. Liance, V. Godot, J. P. Carbillet, D. Meillet, T. Romig, and D. A. Vuitton. 1998). Intestinal and systemic humoral immunological events in the susceptible Balb/C mouse strain after oral administration of Echinococcus multilocularis eggs. Parasite Immunol. 20: 623-9). The infectivity of this batch of eggs was pre-determined by titration-infection experiments in mice, and an infection dose of 2000 eggs was used.

Six week old Balb/c mice were housed in a temperature-controlled light-cycle room with food and water ad libitum. The mice were separated into experimental groups of 10 animals each. Drug suspensions (30 mg/ml NTZ, 10 mg/ml ABZ, and 30 mg NTZ plus 10 mg ABZ/ml, respectively) were prepared in carboxymethylcellulose (CMC) 0.5% (w/v) in water. Drug suspensions were freshly prepared each week and stored at −20° C. for a period of 7 days maximum. The control suspensions containing only CMC were treated identically. Drug- and control-suspensions (100 ml) were applied by intragastric inoculation.

For assessments of secondary infections, parasite tissue was carefully removed from the peritoneal cavity, and the parasite weight was determined as described in: Dai, W. J., and B. Gottstein. 1999. Nitric oxide-mediated immunosuppression following murine Echinococcus multilocularis infection. Immunology 97: 107-16.

For analysis of primary infection, the entire liver was removed, and lesion numbers were determined as described: Siles-Lucas, M., M. Merli, U. Mackenstedt and B. Gottstein 2003. The Echinococcus multilocularis 14-3-3 protein protects mice against primary but not secondary alveolar echinococcosis. Vaccine 21: 431-439.

Comparative statistical analysis was performed by Student's t test, and by ANOVA followed by Tukey comparison test. Analysis was done using the JMPIn statistical package. Differences were considered as significant for p<0.05.

For histological and transmission electron microscopy (TEM) analysis, tissue samples were fixed in 3% paraformaldehyde/1% glutaraldehyde in 100 mM sodium cacodylate buffer (pH 7.2) for 24 h at 4° C., and postfixed in 2% OsO₄ in cacodylate buffer for 4 h at room temperature. The samples were then processed and embedded in Epon 812 epoxy resin as described previously in: Hemphill, A., and S. L. Croft. 1997. Electron microscopy in Parasitology. In: p. 227-268. M. Rogan (ed), Analytical Parasitology, Springer Verlag, Heidelberg. Sections of 1-2 mm in thickness were used for histological examination, and ultrathin sections 80-100 nm for transmission electron microscopy. Sections were loaded onto 300-mesh copper grids, and were contrasted with uranyl acetate and lead citrate as described previously in: Hemphill, A., and S. L. Croft. 1997. Electron microscopy in Parasitology. In: p. 227-268. M. Rogan (ed), Analytical Parasitology, Springer Verlag, Heidelberg. Specimens were viewed on a Phillips 300 transmission electron microscope operating at 60 kV.

For serological and lymphocyte proliferation assays, blood was taken from euthanized mice by heart puncture, and total IgG-, as well as IgG1- and IgG2a-isotype levels against crude E. multilocularis extract (Faub-antigen) and vesicle fluid antigen, were determined according to: Dai, W. J., A. Hemphill, A. Waldvogel, K. Ingold, P. Deplazes, H. Mossmann, and B. Gottstein. 2001. Major carbohydrate antigen of Echinococcus multilocularis induces an immunoglobulin G response independent of alphabeta+CD4+ T cells. Infect Immun. 69: 6074-83.

Splenocyte proliferation assays were carried out essentially as described previously (Dai, W. J., and B. Gottstein. 1999. Nitric oxide-mediated immunosuppression following murine Echinococcus multilocularis infection. Immunology 97: 107-16; and Dai, W. J., A. Hemphill, A. Waldvogel, K. Ingold, P. Deplazes, H. Mossmann, and B. Gottstein. 2001. Major carbohydrate antigen of Echinococcus multilocularis induces an immunoglobulin G response independent of alphabeta+CD4+ T cells. Infect Immun. 69: 6074-83.). Cells were seeded into 96-well round-bottom plates at 2×10⁵ cells/well, and were stimulated either with concanavalin A (ConA; 2 μg/ml) or crude E. multilocularis antigen (50% g/ml), or were left unstimulated as negative controls. All tests were performed in quadruplicates.

### Example 1

The relative ineffectiveness of albendazole or nitazoxanide chemotherapy, when administered separately, was shown in the treatment of secondary infection with E. multilocularis metacestodes, with chemotherapy starting at the timepoint of infection.

The following experimental groups were tested (10 animals/group): (1) non-infected, no treatment; (2) non-infected, NTZ treatment; (3) infected, no treatment (=infection control); (4) infected and treated with CMC (=solvent control); (5) infected and treated with ABZ; (6) infected and treated with NTZ. Balb/c mice were infected by i. p. inoculation of 20 metacestodes in 100 ml HBSS. Treatments were initiated on the same day, and were repeated daily for 35 consecutive days. Mice were euthanized on day 36 following the initiation of drug treatment, and necropsy was carried out immediately thereafter.

Determination of the parasite weight in the different experimental groups showed no difference in parasite weights in the untreated infection control group and the CMC-solvent control group. Parasite weights in treated groups (NTZ treated: 0.17±0.05 g, ABZ treated: 0.08±0.01 g) compared to the solvent (CMC-treated) group (0.23±0.05 g) were lower, however, respective p-values (>0.05) indicated that the corresponding reduction in parasite weight was not statistically significant.

### Example 2

The synergistic effect of combined albendazole/nitazoxanide chemotherapy was shown in the treatment of secondary infection with E. multilocularis metacestodes, with chemotherapy starting at two months post infection. In addition, synergism of combined albendazole/nitazoxanide chemotherapy on parasite ultrastructure is shown.

The following experimental groups (10 animals per group) were used: (1) non-infected and no treatment; (2) infected and treated with CMC (=control); (3) infected and treated with ABZ; (4) infected and treated with NTZ); (5) infected and treated with ABZ/NTZ combination). Balb/c mice were infected as in EXAMPLE 1. Treatments were initiated at 2 months p. i. and were repeated daily for 35 consecutive days. Mice were euthanized on day 36 following the initiation of drug treatment, and necropsy was carried out immediately thereafter.

Analyses of the humoral immune response and of lymphocyte proliferation characteristics indicated no differences between the control and treatment groups.

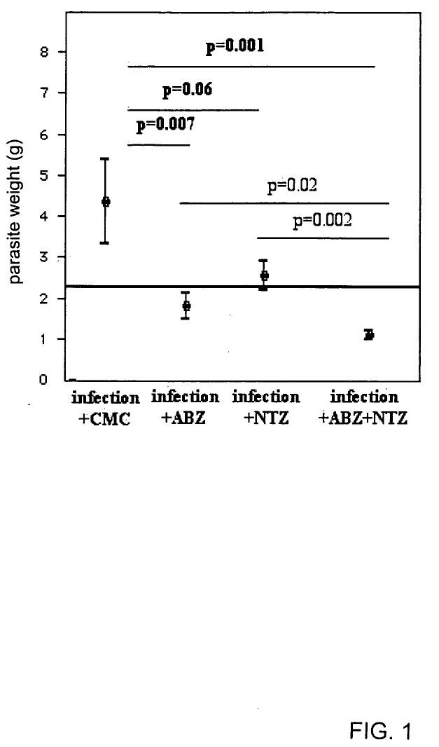

During the time frame of 2 months following infection, the parasite weights within infected control-group mice increased almost 20 times (to 4.3±0.9 g, see FIG. 1). Continuous treatment of mice with ABZ resulted in a significant reduction in parasite weight (p=0.007). Treatment with NTZ alone also reduced the parasite weight, however, the reduction was not statistically significant (p=0.06).

In marked contrast, the combined NTZ/ABZ-treatment showed a pronounced efficacy, with a highly significant reduction in parasite weight (p=0.001) compared to the CMC-control group. Reductions in parasite weights were also significant in relation to the ABZ-treated group (p=0.02) and the NTZ-treated group (p=0.002). In addition, intra-group variations in parasite weights were lowest in the group treated with the NTZ/ABZ-combination, intermediate and similar in the NTZ- and ABZ-treated groups, and highest in the CMC-control group (see FIG. 1).

### Example 3

Nitazoxanide chemotherapy in the treatment of primary infection with E. multilocularis eggs, with chemotherapy starting at two months post infection is shown as a basis for comparison with combined NTZ/ABZ chemotherapy.

Balb/c mice (10 animals) were infected with 2000 E. multilocularis eggs (stored in PBS at 20,000 eggs/ml) by 100 ml intragastric inoculation. Control mice (10 animals) received the identical amount of PBS. Treatments with NTZ were initiated at 2 months p. i., and were repeated daily for 35 consecutive days.

Mice were euthanized on day 36 following the initiation of drug treatment, and necropsy was carried out immediately thereafter.

NTZ had an effect on the development of the parasite in the liver, reflected by the significantly (p=0.01) lower number of lesions detected in the NTZ-treatment group (means of 2.2 lesions/mouse), compared to the infection control group (means of 5 lesions/mouse). Analyses of the humoral immune responses and of lymphocyte proliferation characteristics indicated no differences between the control and treatment groups. Thus, NTZ metabolites are able to reach the actual E. multilocularis target organ, the liver, in concentrations high enough to exhibit anti-parasitic activity, which benefit is therefore also present in the combination chemotherapy of the present invention.

### Example 4

The improved and synergistic effect of combined albendazole/nitazoxanide chemotherapy was shown on the pharmacokinetics of albendazole, nitazoxanide, and their metabolites.

A total of 120 6-week old non-infected Balb/c mice were divided into three treatment groups: group 1 (NTZ—30 mice), group 2 (ABZ—30 mice), group 3 (NTZ/ABZ—60 mice). They received a single dose of 3 mg NTZ (group 1), 1 mg ABZ (group 2) and 3 mg/1 mg of NTZ/ABZ (group 3) in a volume of 100 μl suspended in 0.5% CMC. At defined time points following inoculation, (0, 1, 2, 4, 6, 8, 12, 24 and 48 h), blood samples were obtained from three mice/group, and blood levels of tizoxanide (TIZ), tizoxanide-glucuronide (TIZ-gluc) and ABZSO were assayed.

TIZ and Tiz-gluc were extracted from mouse serum samples using protein precipitation with acetonitrile. The supernatant was evaporated and reconstituted in injection solvent before injection. Chromatographic separation was achieved on a C18 column (5 pm 70×2 mm). Detection was by a mass spectrometer with an ion spray ionization source. Negative ions were detected in multiple reaction monitoring mode (Q1=264 m/z and Q3=216.9 m/z for TIZ, and Q1=439.9 m/z and Q3=263.9 m/z for TIZ-gluc). The lower limits of quantitation were 0.05 μg/ml and 0.2 μg/ml for TIZ and TIZ-gluc, respectively.

ABZSO serum levels were determined by HPLC using a modification of the methods described previously (Ingold, K., P. Bigler, W. Thormann, T. Cavaliero, B. Gottstein, and A. Hemphill. 1999. Efficacies of albendazole sulfoxide and albendazole sulfone against in vitro cultivated Echinococcus multilocularis metacestodes. Antimicrob. Agents Chemother. 43: 1052-1061; Prochazkova. A., M. Chouki, R. Theurillat and W. Thormann. 2000. Therapeutic drug monitoring of albendazole: determination of albendazole, alendazole sulfoxide and albendazole sulfone in human plasma using nonaqueous capillary electrophoresis. Electrophoresis 21: 729-736.). The assay is based upon liquid/liquid extraction of ABZSO at alkaline pH using dichlormethane, a reversed phase C18 Macherey-Nagel Nucleosil column (250/8/4), addition of cyclobendazole as internal standard and solute detection at 230 nm. The mobile phase comprised a mixture of a 5 mM aqueous potassium dihydrogenphosphate (pH is adjusted to 6.5 with a few drops of 20% KOH) and acetonitrile (68:32, v/v). The flow rate was 0.7 ml/min and the temperature was ambient. The assay was based upon extraction from 0.1 ml serum and was calibrated between 0.56 and 8.43 μg/ml ABZSO. The lower limit of quantitation was determined to be 0.3 μg/ml.

FIG. 5 shows the results of the pharmacokinetic study following uptake of a single dose of ABZ, NTZ, and NTZ/ABZ, respectively. Serum levels of NTZ-metabolites TIZ (A), TIZ-gluc (B), and levels of ABZSO(C) were analyzed. Results are presented of means of three mice+/−SD. There was a prolonged persistence of ABZSO in sera of mice undergoing combined treatment in (C).

Analysis of the group receiving a single dose of NTZ revealed that levels of the primary metabolic product TIZ increased rapidly and peaked at 1 h post-inoculation at a mean level of 0.25 μg/ml, and then dropped down to zero at 4-6 h (FIG. 5A). When NTZ was applied in combination with ABZ, the mean TIZ-level at 1 h was clearly higher (0.37 μg/ml), but then decreased more rapidly and reached zero within 2 h. The picture was similar for TIZ-gluc. Corresponding mean levels peaked at 1 μg/ml after 1 h, but were 1.5 μg/ml when applied in combination with ABZ (FIG. 5B). Also, TIZ-gluc-levels decreased more rapidly when applied in combination with ABZ.

In contrast, ABZSO exhibited a slightly lower peak concentration when applied in combination with NTZ (6.5 μg/ml; FIG. 5C). However, during the next 8 h, ABZSO was much more slowly metabolized in the combined treatment group. For instance, at 8 h post-inoculation, ABZSO-levels in the combined treatment group were 5 times higher compared to the group receiving ABZ alone (about 5 μg/ml and 1 μg/ml, respectively). Although in the ABZ-group the ABZSO-level reached zero at 12 h, the serum concentration of ABZSO in the combined treatment group remained 1.5 μg/ml at this time. This example shows that the increased anti-parasitic efficacy of combined NTZ/ABZ-therapy is associated with increased and prolonged ABZSO serum levels for extended periods of time.

Thus, on one hand, the initial uptake of NTZ and conversion to TIZ and TIZ-gluc was enhanced by co-administration with ABZ, resulting in an accelerated degradation of these metabolites (FIGS. 5A, B). On the other hand, the combined treatment enhanced ABZSO-serum levels (see FIG. 5C).

The metabolism of ABZ is similar in mice and humans (Zeugin, T., T. Zysset, and J. Cotting. 1990. Therapeutic monitoring of albendazole: a high performance liquid chromatography method for determination of its active metabolite albendazole sulfoxide. Ther. Drug Monit. 12: 187-190): upon uptake, ABZ is rapidly metabolised to ABZSO, which subsequently disappears at the latest after 10 h (Chinnery, J. B., and D. L. Morris. 1986. Effect of albendazolesulphoxide on viability of hydatid protoscoleces in vitro. Trans. R. Soc. Trop. Med. Hyg. 80: 815-817; and Ingold, K., P. Bigler, W. Thormann, T. Cavaliero, B. Gottstein, and A. Hemphill. 1999, Efficacies of albendazole sulfoxide and albendazole sulfone against in vitro cultivated Echinococcus multilocularis metacestodes. Antimicrob. Agents Chemother. 43: 1052-1061). In contrast to the prior art, combined application of the two drugs in mice increased ABZSO levels up to 5-fold during the time span of 4-8 h following uptake.

Not to be limited by theory, this effect may be based in NTZ and ABZ being metabolized by the same enzyme or enzyme complex wherein NTZ exhibits a higher enzymatic affinity, and because administered at a higher dose compared to ABZ, influences the processing of ABZ and levels of its metabolites. Thus, parasites may be initially exposed to NTZ-metabolites during the first hour after drug uptake, followed by prolonged exposure to increased ABZSO-levels for up to 8 hours after uptake. Thus, parasites may be exposed to the dual action of both drugs. Evidence for this non-limiting mechanism may be found in the transmission electron microscope analysis of EXAMPLE 5.

### Example 5

The synergistic effect of combined albendazole/nitazoxanide chemotherapy on histological and electron microscopical markers of anti-parasite activity.

Histology revealed that no protoscoleces formation took place in any of the mice. In control animals, high numbers of metacestodes were seen, which were often surrounded by host connective tissue, and showed a clearly discernable laminated and germinal layer. Parasite tissue from ABZ- or NTZ-treated mice appeared substantially similar, except that in many areas the vesicles, and the associated laminated layer, were broken and partially fragmented, which was indicative of a limited destructive effect mediated by these drugs.

In marked contrast, metacestode tissue and metacestode residues from mice undergoing combined NTZ/ABZ chemotherapy were mostly encapsulated by dense layers of host connective tissue, although there were also areas with high vesicle density.

Because it was not possible to investigate alterations of the actual parasite tissue on the germinal layer using histology, transmission electron microscopy was performed on sections obtained from the same tissue blocks. For evaluating the ultrastructural changes occurring during drug treatments, it is important to note that the process of tissue damage was not uniform, as it progressed at different rates in different metacestode vesicles. However, within a single metacestode, damage was rather homogenous. Therefore, a large number of different metacestodes, and as a consequence, different areas of a tissue block, were investigated in order to show a complete picture of the tissue alterations induced by ABZ, NTZ, and NTZ/ABZ-treatments.

CMC-control treated parasites exhibited the typical appearance of E. multilocularis metacestode tissue. The outer surface was formed by the acellular, carbohydrate-rich laminated layer, which exhibited variable thickness and a typical, rather amorphous, appearance. The laminated layer is tightly associated with the actual parasite tissue, composed of the tegument, just adjacent to the laminated layer, with microtriches protruding outwards, and distal to the tegument, the inner germinal layer. The germinal layer, although often not well developed and not very prominent in these mice, contained connective tissue cells, muscle cells, few glycogen storage cells, and undifferentiated cells.

In comparison, ABZ-treated metacestode tissue exhibited a number of characteristic alterations. First, in large parts, the microtriches appeared truncated or even absent. Secondly, the parasite tissue was mostly reduced to only the tegument, and the more complex germinal layer was virtually absent. However, occasionally, undifferentiated cells were observed. Thirdly, many areas of the inspected tissue revealed complete breakdown of the tissue integrity.

Similarly, NTZ-treated parasite-tissue also exhibited rather heterogeneous morphological features. Both morphologically intact metacestodes as well as structurally aberrant and largely necrotic parasite tissue were found. A characteristic feature of NTZ-treated parasites was the presence of microtriches even in metacestodes, which appeared to be dying. Closer inspection of the interface between laminated and germinal layer of these severely damaged parasites revealed the presence of numerous vesicles which had been released into the matrix of the laminated layer. Similar accumulation of vesicles in the laminated layer has also been recently observed in studies on metacestodes treated with NTZ in vitro (Stettler, M., R. Fink, M. Walker, B. Gottstein, T. Geary, J. F. Rossignol, and A. Hemphill. 2003. In vitro parasiticidal effect of nitazoxanide against Echinococcus multilocularis metacestodes. Antimicrob Agents Chemother. 47: 467-74).

In marked contrast, the impact of drug treatment was most pronounced in tissue samples obtained from NTZ/ABZ-treated mice. In many areas, the parasite tissue was reduced to the tegument, which did not appear necrotic nor visibly damaged, but was completely devoid of microtriches. In other areas, a few and distorted, undifferentiated cells or glycogen storage cells were seen, but most of the tegument was disintegrated. In addition, many metacestodes exhibited completely distorted genninal layer and tegument, but residual truncated microtriches-like structures could still be discerned, and were seemingly still integrated into the matrix of the laminated layer. Closer inspection of those structures revealed the presence of vesicle-like components embedded into the laminated layer, similar to those observed in tissue of mice which had been treated by NTZ alone. Finally, in many areas only residual laminated layer was observed, which lacked any cellular parasite components, and which was completely encapsulated by host tissue. Thus, the NTZ/ABZ-treatment caused progressive loss of the structural integrity of the parasites, which included severe necrosis of tegument and germinal layer, often accompanied by encapsulation by host tissue.

With respect to the above description, it is to be realized that the optimum formulations and methods of the invention are deemed readily apparent and obvious to one skilled in the art, and all equivalent relationships to those described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

Certain references, patents and other printed publications have been referred to herein: the teachings of each of said publications are hereby incorporated in their respect entireties by reference.

Now that the invention has been described,

1. A method for treatment or prevention of one or more larval or adult stage helminthic infections in a human or an animal subject in need thereof, said method comprising administering to said subject one or more doses of an effective amount of albendazole [5-(propylthio)-1H-benzimidazol-2-yl)]carbamic acid methyl ester or pharmaceutically acceptable salts thereof, in a pharmaceutically acceptable carrier and nitazoxanide (2-(acetolyloxy)-N-(5-nitro-2-thiazolyl)benzamide), or pharmaceutically acceptable salts thereof, in a pharmaceutically acceptable carrier.

2. A method for treatment or prevention of one or more larval or adult stage helminthic infections in a human or animal subject in need thereof, said method comprising administering to said subject one or more doses of an effective amount of a compound of formula (I) or pharmaceutically acceptable salts thereof, in a pharmaceutically acceptable carrier 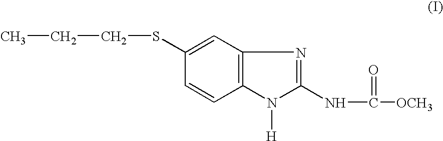
and a compound according to formula (II), 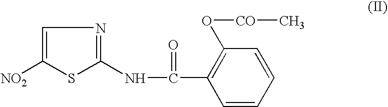
or pharmaceutically acceptable salts thereof, in a pharmaceutically acceptable carrier.

3. The method according to claim 1, said method comprising administering to said subject between about 100 mg and about 3000 mg nitazoxanide and between about 200 mg to about 800 mg albendazole.

4. The method according to claim 1, wherein said nitazoxanide and said albendazole are in a ratio (w/w) of from about 2:1 to about 8:1.

5. The method according to claim 4, wherein said ratio is 5:1.

6. The method according to claim 1, wherein said composition is administered orally.

7. The method of claim 1, wherein said nitazoxanide is in the form of solid particles having a particle size of less than about 200 μm.

8. The method of claim 1, wherein said helminthic infection is a nematode, cestode, or trematode infection.

9. The method of claim 8, wherein said one or more helminthic infection is an intestinal helminth infection.

10. The method of claim 8, wherein said one or more helminthic infection is a larval stage of a cestode selected from the group consisting of Echinococcus multilocularis, Echinococcus granulosis, and Cysticercus cellulosae.

12. A method for increasing serum albendazole sulfoxide levels in a subject undergoing albendazole treatment, said method comprising administering to said subject one or more doses of an effective amount of a composition comprising nitazoxanide, or a pharmaceutically acceptable salt thereof, in a pharmaceutically acceptable carrier.

13. A method for reducing metacestode formation in a subject in need thereof, said method comprising administering to said subject one or more doses of an effective amount of a composition comprising albendazole [5-(propylthio)-1H-benzimidazol-2-yl)]carbamic acid methyl ester and nitazoxanide (2-(acetolyloxy)-N-(5-nitro-2-thiazolyl)benzamide), or pharmaceutically acceptable salts thereof, in a pharmaceutically acceptable carrier.
